(19) United States
(12) Patent Application Publication
(10) Pub. No.: US 2009/0022697 A1
(43) Pub. Date: Jan. 22, 2009

(54) System for the Implantation of Cardiac Staminal Cells with the "Ciuro? Method"

(76) Inventor: Luciano Ciuro, Palermo (IT)

Correspondence Address: Lindsay S. Adams;Day Pitney, 7 Times Square, New York, NY, 10036, US

(21) Appl. No.: 12/224,401

(22) PCT Filed: Aug. 21, 2006
(86) PCT No.: PCT/IT2006/000622

§ 371 Date: Aug. 26, 2008

(30) Foreign Application Priority Data
Mar. 14, 2006 (IT) PA 2006 A 000008

(51) Int. Cl. A61K 35/00; A61M 31/00; C12N 5/02

(52) U.S. Cl. 424/93.7; 604/522; 604/523; 435/374; 604/57

(57) ABSTRACT

The system is composed of two inseparable parts that both make up one Device: I) A container for collecting staminal cells and for their growth factors which is the same as the portcat system with sylicon button to enable the entrance of the Huber needle through a hole. 2) “Active fixation” catheter/s, hollow inside, to permit a rapid flow of the staminal cells into the myocardiac muscular mass and also a rapid flow of the growth factors involved: the various catheters can be fixed to the myocardio by means of an appendix somewhat like a “mouse tail”; they resemble the atrial active fixation catheters that used to be used before the introduction of the J type catheters. The only “but substantial” difference with those mentioned before, is the internal cavity, which should involve even the terminal anchorage part in order to allow the free flowing of any biological material inside the heart without the need for a surgical intervention to open the thorax. The catheters themselves will be connected to the container of the staminal cells nutrition with appropriate watertight attention.

The staminal cells, called totipotents, can be inserted in the cardiac muscle and they can be helped to recharge themselves by a totally new method, which consists in implanting a device positioned in subclavian area RX or LX, and connected to the cardiac muscle with well-positioned cardiac catheters. The use of the system and its installation method require adequate scientific skills due to the potential risk of arhythmicity, both for the technique and for the implanted staminal cells.

The method provides the use of new “active fixation endocarditis” catheters (FIG. 2), which are guided inside the cardiac cavities by appropriate metallic guides, these allow their correct positioning in the specific area with both in phase of forming or stabilized heart-attack scars. The catheters it can be a variable number by necessity. The catheters will be positioned by X-scopia vision with X-band amplifier. They will be hollow inside and in the last part they will be like a “mouse tail” (FIG. 2b), the first part (FIG. 2a) can be screwed by the proper mandrel made of compound material.

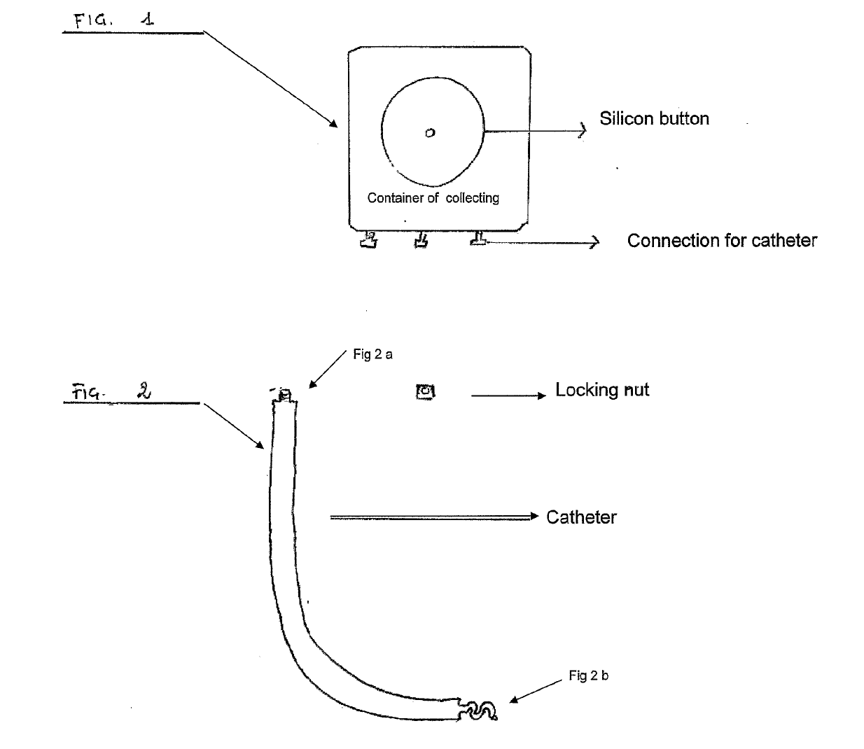

The catheters will be connected to a “container of collecting and nutrition” (FIG. 1), which will be positioned in the right or left subclavian area, and the container will be also fixed by wires of silk suture 3-0 to the pectoral muscle, after having created an adequate space (pocket of the container of collecting and nutrition).

The system provides the canalization of the subclavian vein, by introducturies like Cordis and Seldinger (number of FR to be decided according to the case).

One or more catheters will be inserted into the right cardiac space, while in the left by puncture inter-atrium or coronary sinus. At this point we can proceed at the active fixation in the heart muscle by the mandrel which allows the screwing of the last part of the catheter to the heart muscle; the potential lesions will be collected and the resistances in OHM will be measured. In the end we should guarantee if the catheter is well fixed through the “traction proof”. Every operation will be registered by videorecorder or cd. With this ends the first part of the implantation.

The second part begins by making a pocket of different sizes, in relation to the thorax of the patient.

At this point the container mentioned above will be implanted into the pocket by suture silk wires which pass through a sort of slot that belongs to the external covering of the container (from 4 to 6).

The container is made of two sectors, in the one below there is the real cells collecting space; in the one above there is a watertight “button” in silicon or composite material, with a hole for the insertion of the Huber needle. The space below has to be connected to the catheters which are implanted by “locking nut” system with spring-lock and watertight security system.

The container of nutrition will be made of steel or bio-compatible material.

The third phase requests, first to close the pocket and then the skin tissue (cutis derma) by deep suture in VYCRYL and superficial suture in silk.

The staminal cells might be injected in any time by the Huber needle together with the nutrition and the growth factors. Then, it follows the Ecocardiographic control by trans esophageal.

The system must be lubricated continuously (at least 3 times within 24 hours after the implantation) by physiological solution adequately heparinated with 1 or 2 cubic centimetres of ready sodic heparin.

The implantation can be performed on the seventh day after the heart-attack; in case of the clear enlarging cardiomyopaties at any time, it is preferable that the primary PTCA or rescue with or without “stent” apposition has already happen. The cells must be selected by an adequate Centre and they has to be of two kinds: neoangiogenetic or muscular with dna map method by this time habitual. The cells have to be marked out with monoclonal antibodies (or radiomarked). The growth of the cells has to be followed by the ordinary cardiologic system, such as (ECG, ECOCARDICOLORDOPPLER, MYOCARDIC SCINTIGRAPHY).

The system with his complexity, can be removed in any time.

1. I claim the total invention of the method and the resulting device created in order to implant staminal cells through the heart: the system is composed of two inseparable parts: the container for the collecting and for the nutrition of the staminal cells which is connected to the cardiac muscle by “active fixation” catheters, hollow inside. Furthermore I claim the total application of the implanting method which it is still unknown to the scientific world.
